(19) United States
(12) Patent Application Publication
(10) Pub. No.: US 2019/0184393 A1
(43) Pub. Date: Jun. 20, 2019

(54) MICROFLUIDIC DEVICE

(71) Applicant: ExVivo Labs Inc., Waterloo (CA)

(72) Inventors: Md. Almostasim MAHMUD, Oshawa (CA); Eric BLONDEEL, Waterloo (CA); Brendan MACDONALD, Oshawa (CA)

(73) Assignee: ExVivo Labs Inc., Waterloo (CA)

(21) Appl. No.: 16/323,226

(22) PCT Filed: Aug. 18, 2017
(86) PCT No.: PCT/CA2017/050979

§ 371 Date: Feb. 4, 2019

Related U.S. Application Data
(60) Provisional application No. 62/377,236, filed on Aug. 19, 2016.

(51) Int. Cl. B01L 3/00; G01N 33/48; B32B 37/12; B32B 38/10

(52) U.S. Cl. CPC B01L 3/502707; G01N 33/48; B01L 2300/0887; B32B 38/10; B32B 37/12

(57) ABSTRACT

A method and system for subtractive patterning of a substrate, which is utilized in the making of paper-based microfluidic analytical devices (pPADs). By adhering the substrate on an impermeable backing material, the substrate is etched to yield high resolution features, which can be utilized to construct MPADS capable of flowing and testing extremely small sample volumes. This system and method can be modified for various substrates to construct features for two and three dimensional flow systems. A substrate assembly is formed by affixing a substrate layer (e.g. paper) to an impermeable layer (e.g. foil). Portions of the substrate layer are cut away using an etching device to form one or more subtractive patterns on the substrate assembly the define fluid flow regions.

### FIELD OF THE INVENTION

The present invention relates generally to microfluidic devices, and more particularly, to such devices which comprise high resolution subtractive patterning. The present invention also relates to a system and method to making such microfluidic devices.

### BACKGROUND OF THE INVENTION

Analytical assays are useful in diagnostic applications, for example, in human health (e.g. blood and urine testing), environmental contamination (e.g. water and soil testing) and industrial food and drug preparation (e.g. bacterial contamination testing), but often require large and costly laboratory instruments and trained operators.

Paper-based tests that flow in one direction (i.e. one dimensional, 1D), such as lateral flow immunoassays, have been in use for some time for various applications (e.g. home pregnancy tests). They are functionally simple, disposable and require little instruction on the part of the user to operate. This field has become more diverse with the advent of paper-based microfluidic analytical devices, termed “μPADs”, which can perform more complex tests, as well as parallel multiplexing tests, in multiple flow directions (i.e. two and three dimensions, 2D & 3D) with narrower flow channel dimensions (and by extension smaller required sample volumes), than the common paper strip tests of the past. The ability to work with smaller volumes is important when testing samples that are difficult to acquire in large volume such as point-of-care tests for human health.

This field of μPADs began with processes of additively applying hydrophobic treatments to hydrophilic substrates, patterning these treatments in various geometries for flowing microscale volumes of samples (i.e. paper-based microfluidics). Later, additive methods also included hydrophilic treatments to hydrophobic substrates using methods such as photolithography with a photo-mask to achieve patterning, or various printing technologies to apply the necessary chemicals in the appropriate pattern. Several of the printing methods, such as wax printing, could not achieve high-resolution patterning because of lateral bleeding of the applied chemical in the paper, making fine micro-features impossible, hampering the design and reproducibility of μPADs created using these methods. Other methods, such as those utilizing photolithography, maintained higher resolution, but required relatively expensive manufacturing methods and/or exotic chemicals.

Following the development of additive methods, researchers subsequently investigated subtractive methods utilizing machine-controlled cutting instruments, as well as etching tools like lasers to create hydrophilic regions bound by gaps in the absorbent substrate that fluid could not cross. However, these “cut-out” methods produced products that were fragile and difficult to handle. Furthermore, these methods were limited with respect to how small the feature sizes could be made (e.g. channel widths and other geometries) and therefore the minimum sample volume for such cut-out paper-based microfluidic tests was higher than the sample volume required for the additive methods. Finally, these cut-out methods were limited with respect to scalability of the process for manufacturing.

Thus, it is desirable to develop paper-based microfluidic analytical devices that overcome one or more disadvantages of prior such devices.

### SUMMARY OF THE INVENTION

A novel system and method for subtractive patterning of an absorbent substrate to yield a novel microfluidic device has now been developed.

According to a first aspect of the invention, a microfluidic device is provided comprising a substrate layer, a layer impermeable to an etching device; and optionally, an adhesive layer for affixing the substrate layer to the impermeable layer, wherein portions of the substrate layer are removed to form a subtractive pattern suitable to direct fluid flow within the device.

According to another aspect of the present invention, there is provided a microfluidic device including a paper layer, a foil layer impermeable to an etching device; and an adhesive layer for affixing the paper layer to the foil layer. Portions of the paper layer are removed to form subtractive patterns on the microfluidic device.

In another aspect of the invention, method of manufacturing a microfluidic device is provided, the method including forming a substrate assembly, the substrate assembly comprising a substrate layer, an impermeable layer and optionally, an adhesive layer; cutting away portions of the substrate layer using an etching device to form one or more subtractive patterns on the substrate assembly; and cutting the substrate assembly using a cutting device into one or more microfluidic devices.

There is further provided a system for manufacturing microfluidic devices, the system including a feeder assembly, for directing a layer of substrate and a layer of impermeable material towards a combining assembly for affixing the layer of substrate to the layer of impermeable material to form an assembled substrate; an etching device for cutting away portions of the layer of substrate to form one or more subtractive patterns on the assembled substrate; and a cutting device for cutting the assembled substrate into one or more microfluidic devices.

The present invention provides microfluidic devices comprising high-resolution subtractive patterning of an absorbent substrate coupled with an impermeable backing, which is durable, and results in a device desirably comprising small feature sizes which may advantageously be used in low volume fluid tests (e.g. using microliter-sized samples, such as samples of less than 1000 μL, and preferably less than 10 μL, including samples of less than 1 μL).

Further, the present invention does not require expensive or exotic manufacturing methods or materials, and the process is readily scalable for mass manufacturing.

Other features and advantages of the present invention are described more fully below.

### BRIEF DESCRIPTION OF THE DRAWINGS

Embodiments of the present invention will now be described, by way of example only, with reference to the attached Figures:

FIG. 1 is a schematic illustrating a) the layers of a microfluidic device in accordance with an embodiment of the invention, b) the use of a laser for subtractive patterning within the device, and c) an expanded view of the subtractive patterning.

FIG. 2 is a schematic illustrating the manufacture of microfluidic devices according to an embodiment of the present application.

FIG. 3 is a graph illustrating the utility of hydrophobic barriers prepared at various powers and cutting speeds of a laser.

FIG. 4 graphically illustrates the average barrier width in aluminum foil backed Whatman 1 chromatography paper when utilizing a laser for subtractive patterning at different %-Power and %-Speed.

FIG. 5 illustrates high resolution microchannel widths in aluminum foil backed Whatman 1 chromatography paper when utilizing a laser for subtractive patterning.

FIG. 6 illustrates an eight-way multiplex μPAD architecture useful in a multiplex assay.

FIG. 7 is a schematic illustrating a) process for making a microfluidic device comprising multiple channels, and b) the multi-channel device.

FIG. 8 illustrates the methodology utilized to test a microfluidic device.

FIG. 9 illustrates the utility of fluid flow channels of various widths in a microfluidic device according to an embodiment.

FIG. 10 graphically illustrates the effect of substrate fiber width on utility of fluid flow channels in a microfluidic device according to an embodiment of the invention.

FIG. 11 graphically illustrates flow distance through various channel widths formed in a) Chr-1, b) 3 mm Chr and c) RC-55 paper substrates, respectively.

### DETAILED DESCRIPTION OF THE INVENTION

The present invention provides a microfluidic device comprising a substrate layer affixed to a layer impermeable to an etching tool, wherein portions of the substrate layer are removed to form a subtractive pattern which directs fluid flow within the device.

A system and method are also provided for manufacturing a microfluidic device having high resolution subtractive patterning of the substrate. As used herein, the term “high resolution” refers to subtractive patterning capable of creating features with sizes less than 200 μm. The device is made by adhering the substrate to an impermeable backing material, followed by etching of the substrate with a suitable etching tool to yield high resolution features. The system and method may be utilized to construct paper-based microfluidic analytical devices (μPADs) useful for testing sample volumes of any size, including extremely small fluid sample volumes (e.g. microliter-sized samples, such as samples of less than 1000 μL, and preferably less than 10 μL, including samples of less than 1 μL). This system and method can be modified for various substrates and impermeable layers as herein described, and to construct devices of various geometries and dimensions, including two and three dimensional flow systems.

According to the present invention, an assembly of a layer that is impermeable to an etching device (e.g. a laser-impermeable backing) affixed to a substrate is provided. The assembly comprises a substrate layer and an impermeable backing layer, and if required, an adhesive layer. The substrate layer comprises a material that is penetrable by the selected etching tool such that a subtractive pattern in which portions of the substrate layer are removed may be formed in the substrate layer with the etching tool. The substrate layer may be any absorbent material that is permeable to (or penetrable by) an etching tool, and which is hydrophilic. In one embodiment, the substrate layer is a paper layer, such as cellulose chromatography paper. In other embodiments, the substrate may be made of another material. For example, the material of the substrate layer may be, but is not limited to, glass fibre paper, nitrocellulose, blotting papers, polymers, or plastics. The material of the substrate layer may be of varying thicknesses and may have various pore sizes. Other possible absorbent substrates may be used as the substrate layer according the present invention.

The impermeable layer may be any material that is impermeable to (or not penetrable by) a selected etching tool such as a cutting laser, or any precision-focused cutting tool. The laser may be any CO₂ laser, or may be another type of laser, such as for example, a gas laser, a chemical laser, a dye laser, a metal-vapor laser, a solid-state laser, or a semiconductor laser. The etching tool may also be a plasma cutting tool or may be a water-jet cutting tool.

In one embodiment, a metallic foil may be used as the impermeable layer (e.g. copper foil, tin foil, iron foil, steel foil, aluminum foil, etc.). In this regard, a suitable foil will have a thermal conductivity that renders it to be impermeable under the parameters of the etching tool to be used. A preferred impermeable layer is aluminum foil. Aluminum foil has the characteristics of being thin (e.g. approximately 10-50 μm) and flexible, which facilitates roll-to-roll manufacturing of the present devices, as well as facilitating the use of the resulting device in a skin patch. Other impermeable layers that may be utilized include material coated with an impermeable layer, for example, paper coated with a metallic layer, a wax layer or polymer layer. The impermeable layer may also comprise an inflexible material having a thickness that may be greater than that of aluminum foil. For example, the impermeable layer may be a plastic or polymeric material, e.g. polyethylene or polymethylmethacrylate.

As will be appreciated by one of skill in the art, the impermeable layer may vary with the etching tool utilized. More particularly, a layer which is impermeable to one etching tool may not be impermeable to another etching tool. Or, a layer which is impermeable under one set of parameters (e.g. low power or high speed) of a given etching tool may not be impermeable to a different set of parameters (e.g. high power or low speed) for the same etching tool. For example, a wax paper is suitable as an impermeable layer with a low powered etching tool, while a metallic foil layer is a suitable impermeable layer at much greater power levels.

The substrate layer is affixed to the layer impermeable to an etching tool. These layers may be affixed naturally, without the addition of an adhesive, due to an inherent adhesive property of one or both of the substrate and impermeable layers. An example of a self-adhering impermeable layer is wax paper.

Alternatively, the substrate layer is affixed to the impermeable layer with an adhesive layer. The adhesive layer may be any adhesive material suitable for adhering the selected substrate layer to the impermeable layer. For example, the adhesive layer may be an adhesive tape (including a double-sided tape), a pressure sensitive adhesive, an adhesive wax, or any suitable glue product. The adhesive layer is applied according to established techniques to either the substrate layer, the impermeable layer or both, in amounts sufficient to achieve adherence of the substrate to the impermeable layer.

The shape and size of the present microfluidic device is not particularly restricted, and may be any shape and size suitable for the utility for which it is intended. Thus, the device may be prepared sized for use in a hand-held device, or may be prepared in smaller or larger sizes based on the intended utility of thereof.

In a specific embodiment, as exemplified herein, the microfluidic assembly may be formed with aluminum foil impermeable layer applied to a paper substrate with adhesive tape. However, it is to be understood that a variety of potential substrates, backings, adhesives, arrangements of layers including multi-layer and double-sided systems, and multiple μPAD geometries may be prepared in accordance with embodiments of the present invention.

The present microfluidic device comprises a subtractive pattern that directs fluid flow within the device. The subtractive pattern is formed within the penetrable substrate layer using a selected etching device. The subtractive pattern is a portion or region of the device in which the substrate has been removed to expose the impermeable layer and provide a hydrophobic barrier region which does not permit fluid flow (e.g. which is non-absorbent). The subtractive pattern is generally shaped to provide a region of the substrate layer which is a hydrophilic fluid flow region, i.e. the hydrophobic barrier region surrounds or encompasses the hydrophilic fluid flow region (e.g. an absorbent region). For example, the subtractive pattern may provide one or more hydrophilic sample regions or zones (e.g. in any desired shape such as circular, oval, square or other geometric shape, or an irregular shape) within the substrate layer onto which a sample may be applied. The subtractive pattern may be further formed in the substrate such that the sample zone is connected to one or more hydrophilic detection or readout zones via one or more hydrophilic channels that permit fluid flow from the sample zone to the detection zone (e.g. for example, the subtractive pattern may provide an hourglass-shaped fluid flow region in the case of a single detection zone, or a shape comprising a central sample zone with multiple appendages extending therefrom in the case of two or more detection zones).

The hydrophobic barrier region is sized to prevent fluid flow from the adjacent hydrophilic fluid flow region (e.g. the sample, detection or channel zones). Preferably, the barrier region is minimally sized to maintain the device as compact as desired. However, the barrier region must not be so small that bleeding of fluid occurs across the barrier and into substrate on the other side of the barrier. The hydrophobic barrier may, for example, be less than 100 μm wide, preferably less than 80 μm, 70 μm, 60 μm or 50 μm wide, and greater than 25 μm wide, preferably greater than 30 μm, 35 μm or 40 μm wide. A preferred width of the barrier region is in the range of about 25-80 μm, 25-55 μm, or 30-50 μm, or 35-45 μm.

Regarding hydrophilic channels that permit fluid flow within the substrate, for example, between a sample zone and a detection zone, it has been determined that the suitable channel width varies with the substrate material, and in particular, the width of the fibers of the substrate material. The larger the width of the substrate fibers, the larger the width of the channel that permits fluid flow. To facilitate fluid flow, the fiber structure of the hydrophilic channel is preferably continuously linked along the channel pathway to assist with wicking of fluid along the channel by capillary forces. In some cases, channel widths of less than 100 μm are possible in substrates with average fiber widths of less than 5 μm, such as fiber widths of less than 2 μm, or 1 μm, for example, but not limited to, 0.1-0.5 μm. Substrates comprising fibers of an average width greater than 5 μm, such as 10-20 μm, preferably comprise channels of greater than 100 μm, e.g. 110 μm, 120 μm, 130 μm, 140 μm, 150 μm and greater.

The present microfluidic device, comprising a hydrophilic fluid flow region or regions, is useful in a variety of applications. A fluid sample may be introduced to the sample zone in the device, and will flow within the fluid flow region to one or more detection or test zones. The detection or test zones may include one or more reagents reactive with or useful to detect a target component within the sample zone. Examples of such applications include, but are not limited to, biomedical diagnostics such as pregnancy tests, glucose tests, biomarker tests, etc.; environmental testing such as water testing for microbial or other contaminants (e.g. arsenic); and any complex geometric high resolution architecture for holding a sample. Thus, fluid samples that may be analyzed using the present device include, but are not limited to, water or water-containing samples from various sources (e.g. tap, well, pond/lake, wastewater, rainwater, etc.), and bodily fluids such as blood, urine, saliva, sweat, tears or amniotic fluid.

Sample volumes for use with the present device may vary. Advantageously, the present devices may be sized to accommodate sample sizes in the microliter range, such as samples of less than 1000 μL, and preferably less than 10 μL, including samples of less than 1 μL.

The subtractive patterns described and illustrated herein are exemplary only and other feature patterns may be printed on the substrate. In the fabrication technique of the present invention, the etching tool, such as a laser, is used under conditions and parameters sufficient to cut through a selected substrate, generating hydrophobic barriers along the cut line, but not penetrating or cutting through the impermeable layer. Thus, the impermeable layer provides a continuous support for the microfluidic device and enables the cutting of microscale features with narrow hydrophobic barriers in the substrate layer.

The present device may be provided as an individual device, or in other configurations such as a multi-layer device, a double-sided device, or a multi-dimensional device. A double-side device comprises two devices adhered back to back, or sharing the same impermeable layer with a substrate layer on both sides thereof, such that a subtractive pattern (either the same or different pattern) exists on both sides of the device. Multi-layer devices comprise 2 or more substrate and impermeable layers to provide subtractive patterns at different levels, for example, for different diagnostic utilities. Multi-dimensional devices comprise 2 or more devices connected via channels which permit fluid flow from one device to another. Such fluid flow channels, thus, connect the fluid flow region of a first device with the fluid flow region of a second, third or more devices. As one of skill in the art will appreciate, various configurations of fluid flow between multiple devices may be accommodated. Fluid flow channels comprise a material that permits flow of fluid, including a substrate material as above-described, which may be provided on a support.

In another aspect of the invention, a simple fabrication method that enables subtractive patterning of compact and microscale features on microfluidic devices, such as paper-based microfluidic devices, is provided. The patterning is achieved using an etching tool. For example, a manufacturing line may be used to assemble an impermeable layer with a substrate (such as a paper layer). If either or both of the impermeable layer and substrate are self-adhering, then the assembly may simply comprise press-fitting. If not, then the method includes application of an adhesive to one or both of the impermeable and substrate layers, followed by assembly of these layers. Next, the subtractive patterning may be performed on the substrate-side of the assembled substrate using the etching tool under conditions and parameters suitable for the selected substrate and impermeable layer. The etching tool is utilized to remove small areas of the substrate to expose the impermeable layer, e.g. aluminum foil backing, producing a subtractive pattern. The etching tool may be a laser. The adhesive layer prevents movement of the substrate relative to the impermeable layer to yield etched boundaries that are uniform and consistent in the microfluidic device (μPADs). Once the subtractive patterning of the substrate is completed, a cutting machine may be utilized to cut the etched assembled substrate into multiple microfluidic devices. It is to be understood that a variety of substrates, impermeable layers, etching tools and other system features are contemplated, and that the power and speed settings may vary accordingly.

In the fabrication method of the present invention, the barrier width for restricting the flow within an absorbent substrate may be modulated by the speed of the etching tool used to remove sections of substrate, as well as the power of the etching tool in the case of a laser for subtractive patternings. As used herein, the barrier width is the width of the vacant hydrophobic region of the device resulting from the removal or subtraction of substrate from the assembled substrate (i.e. a region in which the impermeable layer is exposed). For example, one or more circular μPAD designs (e.g. 3 mm diameter) may be made at a range of speed and power settings for a laser etching tool. For example, a barrier width of 39±15 μm may be achieved at 3% power setting and 0.75% speed settings for the laser etching tool. In another example, one or more square μPAD designs may be made at a range of speed and power settings for a laser etching tool. For example, a minimum barrier width of 36±13 μm may be achieved at 3% power setting and 0.75% speed settings for the laser etching tool. The example barrier widths above-described are achieved using the speed and power of a laser etching tool in subtractive patterning on a paper substrate, for example, a Whatman1 chromatography paper substrate.

An example manufacturing line for producing microfluidic devices in accordance with the invention as shown in FIG. 2, can be equipped with all elements for large scale continuous production of the present microfluidic devices. In terms of the mass production capability, the fabrication process may include, (i) a feed system for the substrate and impermeable layers, (ii) a system for affixing the substrate and impermeable layers, (iii) a laser cutting system, and (iv) a system for cutting the final devices (e.g. press cutting). Alternatively, affixing of the substrate and impermeable layers may be performed as a separate process from the manufacturing line. As shown, the substrate and impermeable layer feed systems feed a sheet from the substrate roll and a sheet from the impermeable layer roll into the system. In some embodiments, the system also includes means for applying adhesive layer onto one or both of the substrate and impermeable layers being fed into the system. The system for affixing the substrate and impermeable layers may include a plurality of rollers to adhere the substrate layer to the impermeable layer. The laser cutting system may include a laser for removing small areas of the substrate to expose the impermeable backing, thereby producing a subtractive pattern. The optional adhesive layer prevents the substrate from moving making the etched boundaries stable within the paper-based microfluidic devices (μPADs). Once the subtractive patterning of the substrate is completed, the system for cutting the final devices (e.g. a cutting machine) may be utilized to cut the substrate into individual microfluidic devices.

Advantageously, the materials required to manufacture the present microfluidic devices are inexpensive, readily available and easy to use in the present fabrication process. In addition, the assembly and fabrication method of the present invention can be utilized for the mass production of μPADs, contributing to the efficiency of making the present devices. Further, the fabrication method enables miniaturizing of μPADs so that micro-sample volumes can be used, thereby reducing the amount of material used in the device, the chemical reagent volumes required for bioassays, the packaging costs, to result in inexpensive μPADs for global use in diagnostic and environmental testing applications.

Embodiments of the invention are described by reference to the following specific examples which are not to be construed as limiting.

### Example 1

A laser cutting fabrication technique was used to prepare a microfluidic device comprising chromatography paper (Whatman, 1 CHR) backed with aluminum foil to create small precise features.

Materials and Chemicals—

Aluminum foil (Diamond-Reynolds Consumer Products Inc., thickness: 15 μm) and double-sided adhesive tape (Studio) were utilized. The cellulose chromatography paper (Whatman grade 1 CHR by GE healthcare, size: 20 cm×20 cm, thickness: 0.18 mm) and artificial urine sample with glucose (Water >98.89%, glucose 1%, Methylparaben 0.1%, Alizarin Yellow 0.0035%, Thymol 0.0017%) were purchased from VWR International (Mississauga, Ontario, Canada). The red dye (Allura Red AC dye content 80%), deionized water, glucose oxidase (Aspergillus niger), horseradish peroxidase (HRP) and potassium iodide were purchased from Sigma-Aldrich (Oakville, Ontario, Canada). Solutions were made using the deionized water. The coloured dyes were extracted from colour markers (felt-tip pens) manufactured by Studio.

Fabrication of Micro Features—

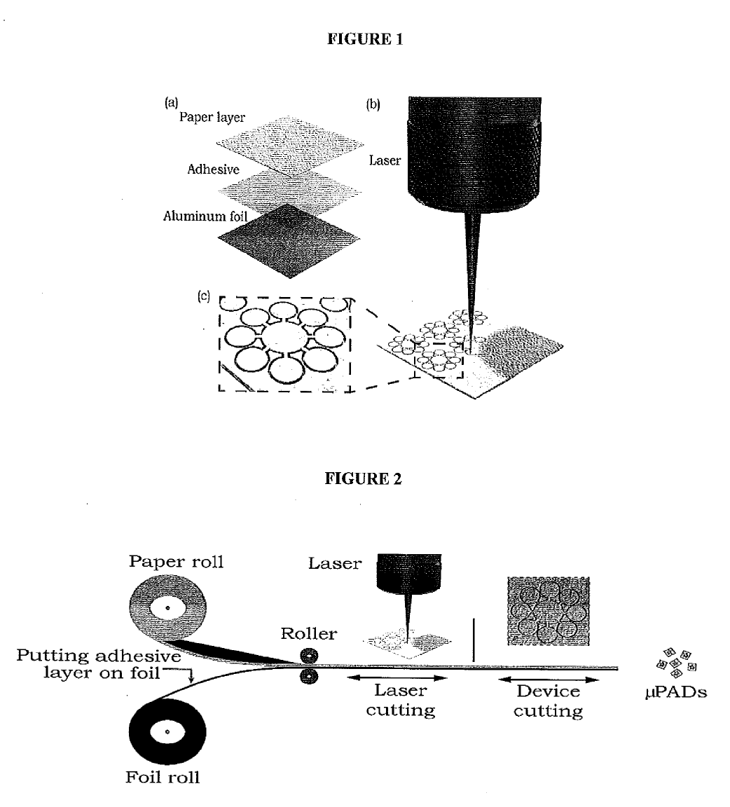

In order to fabricate the compact and microscale features, a paper-based device comprising chromatography paper backed with aluminum foil was assembled, as shown in FIG. 1a. The aluminum foil was affixed to the paper with double-sided tape or by gluing the foil to the paper layer, or by using foil tape. The desired feature patterns were drawn on a PC using InkScape software. These patterns were printed onto the foil backed paper using a 30 W CO₂ laser with a wavelength of 10.6 μm (Speedy 100, Trotec), as shown in FIG. 1b. The foundation of the fabrication technique is that a 30 W laser beam can cut through the paper layer (and adhesive layer), generating channels with hydrophobic barriers where the material is removed, but cannot cut through the aluminum foil layer, as illustrated in FIG. 1c. In order to cut through aluminum, a laser power of approximately 1000 W is typically required. The foil backing thus provides a continuous, durable support for the paper-based microfluidic device, which fixes the paper layer in place and enables the cutting of precise microscale features with narrow hydrophobic barriers in the paper. Since the foil layer is adhered to the paper, the final device will not suffer from any shifting of the microscale features and is readily handled while testing.

The feature and barrier sizes were measured using a USB microscope (xcsource, 20×-800×, 8 μLED, 3D Digital Zoom Microscope) with Toupview software. The assay images were captured using a DSLR Camera (Nikon D5200 with Nikon Af-s Dx Micro 40 mm F2.8G lens) and a scanner (RICOH, Aficio MP 2002). A JEOL 6400 scanning electron microscope (SEM) was used to take micrograph images of the chromatography paper.

Capability for Mass Production—

In terms of the mass production capability, the present fabrication process includes: (i) a paper and foil feed system, (ii) affixing of the paper and foil, (iii) a laser cutting system and (iv) cutting the final paper devices (e.g. press cutting). A single manufacturing line can be equipped with all these facilities for large scale continuous production as shown in FIG. 2. The paper and foil may also be pre-affixed prior to the above process.

Assay Testing with Dyes and Glucose—

The present microscale devices were tested by performing a dye test and a glucose test on devices with eight test readout zones using only 2 μL of sample fluid. For the dye test, approximately 0.2 μL of each of the eight different colour dyes (marker ink) were spotted in the test readout circles and allowed to dry at room temperature. Yellow coloured marker dye (2 μL) was then placed on the sample zone, which flowed through the channels to the readout zones. For the glucose test, 0.1 μL of 0.6M potassium iodide was spotted on the test readout zones followed by 0.1 μL of glucose oxidase-horseradish peroxidase (120 units of glucose oxidase and 30 units of horseradish peroxidase per mL of solution) using a standard procedure (Martinez et al. Anal. Chem., 2008, 80, 3699-3707). These were allowed to dry at room temperature. Artificial urine with glucose (2 μL) was then placed on the sample zone, which flowed through the channels to the eight readout zones.

### Results

Smallest Width of the Hydrophobic Barrier—

To optimize the barrier width, circles of 3 mm diameter were fabricated on the foil backed paper sheet for a range of laser powers and cut speeds. Narrower barrier widths enabled inclusion of more patterns on a single device, since they can be packed more closely together, which gives the capability to perform more tests using smaller sample fluid volumes. However, if the barriers are too small then there may be bleeding of the fluid across the barriers. A higher laser power removes a larger area of the paper and thus generates larger cut widths. Similarly, lower speeds for the laser cutting head results in the removal of more paper material and larger cut widths. The power and speed are adjustable as percentages of the maximum values, where the maximum power for the laser is 30 W and the maximum cut speed is 80 cm/sec. A series of circular patterns were cut on a single sheet, as shown in FIG. 3, beginning with a speed of 0.5% and a power of 1% (of the maximum speed and power) and increased by increments of 0.25% for speed up to a maximum of 3%, and 1% increments for power up to a maximum of 8%. The circles were tested for cross barrier bleeding by placing 0.6 μL of red dye for each of the power and speed combinations, as shown in FIG. 3. The dotted line in FIG. 3 separates the successful and unsuccessful circles such that circles above the dotted line exhibited cross barrier bleeding and those below did not.

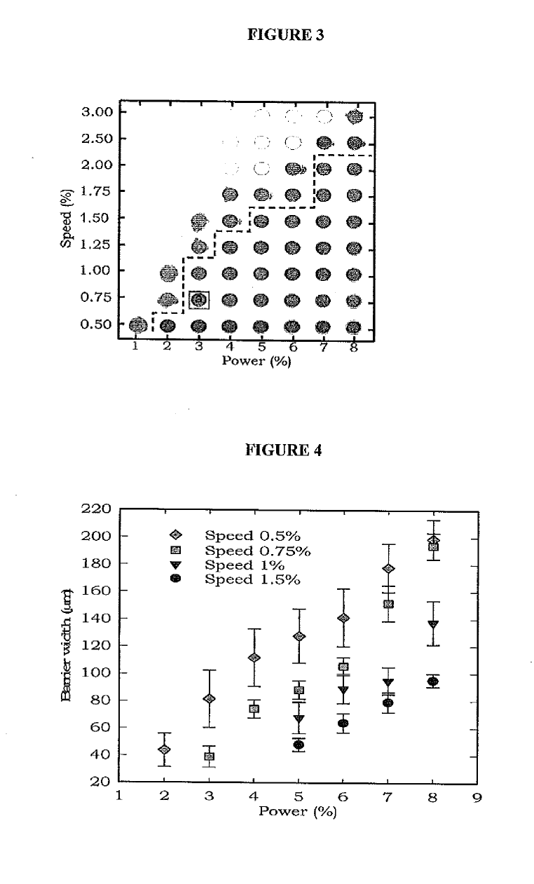

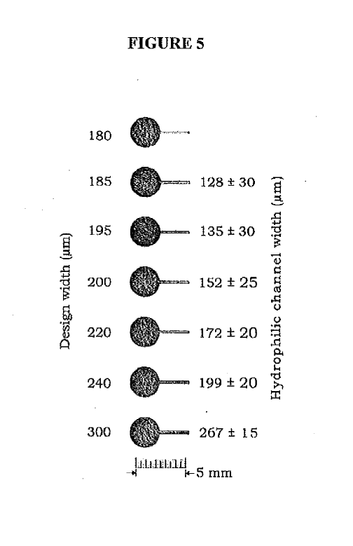

To determine which of the successful circles had the narrowest barrier width, the barriers of each circle were measured by analysing microscope images and plotting the results in FIG. 4. In this non-limiting embodiment, the narrowest barrier was 39±15 μm, resulting from a speed of 0.75% and a power of 3%, which is shown as the boxed circle in FIG. 3. For comparison, the previously reported barrier width for laser cutting was 400 μm in filter paper (as described in Nie et al. Analyst, 2012, 138, 671-676) and for laser etching was 85±5 μm in nitrocellulose membranes (as described in Spicar-Mihalic et al. J. Micromech. Microeng., 2013, 23, 067003).

FIG. 4 also confirms that slower speed values and high power values result in thicker barriers. It can also be seen from the results in FIG. 3 and FIG. 4 that a wide range of laser power and speed combinations can be used depending on what size of hydrophobic barrier is required for each application.

Similar tests were performed for 3 mm square μPAD designs that were made at a range of speed and power settings for the laser etching tool. The narrowest barrier width in this test was determined to be 36±13 μm, resulting from a speed of 0.75% and a power of 3%.

The system and method of the present invention provides barrier widths less than conventional solutions. The system and method of the present invention provides barrier widths less than than 55 μm, and preferably less than 39 μm, and more preferably equal to or less than 36 μm. Smaller barrier widths may achieved by the present invention depending on one or more of the type of substrate used, the power of the etching device, the speed of the etching device and the focusing capability of the etching device.

Smallest Width of the Paper Channels (Hydrophilic Pathways)—

To determine the smallest possible features that can be created using this technique with Whatman 1 CHR paper, 3 mm long channels of different widths connected with a 3 mm diameter circles were designed, as shown in FIG. 5. In FIG. 5, shown are the line to line distances that were drawn for the pattern in the software (left side), and the actual widths measured with the USB microscope (right side). From FIG. 5, an approximate reduction in channel width is seen ranging from 33 to 60 μm between the drawn distances and the actual channel widths. Each channel was tested by placing 0.7 μL of red dye in each circle and observing if the fluid could flow down the whole length of the channel. The smallest paper channel had an actual width of 128+/−30 μm. Close inspection of the paper channels revealed that at widths below this value the fibers in the fibrous matrix were becoming loose and had lost their ability to remain woven with neighbouring fibers to provide a continuous path for the fluid. Thus, it was determined that a paper channel width of at least about 100 μm is sufficient to allow the fibers to remain part of the fibrous matrix for this paper type. To see if this length scale corresponds to physical parameters, SEM images of the chromatography paper were examined and fiber widths as large as 20 μm and gaps between fibers as large as 50 μm were observed for this paper type.

Thus, system and method of the present invention provides channel widths less than conventional solutions. The system and method of the present invention provides channel widths less than 270 μm, preferably less than 150 μm, and more preferably equal to 128 μm±30 μm. Smaller channel widths may achieved by the present invention depending on one or more of the type of substrate used, the power of the etching device, the speed of the etching device and the focusing capability of the etching device. For example, the minimum channel width may vary as different substrate materials may have different thresholds for breakdown (e.g. the minimum channel thickness before the substrate breaks down).

Dye Test with Small Sample Volume—

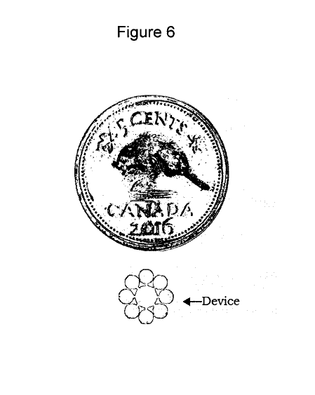

A device was prepared using the above foil-backed laser cut method with a sample circle in the middle (diameter of 3 mm), which fed eight test readout zones (diameter of 2 mm) connected by channels that were 280 μm long with a design width of 300 μm, and a barrier width of 39±15 μm, as shown in FIG. 6, demonstrating the potential for use of the present device in multiple assays from a single sample volume. Using the fabrication technique of the present invention, the circle diameters could be made much smaller and are only limited by the accuracy of the experimenter pipetting the sample (e.g. for the sample circle diameter) and the ability for naked eye detection (e.g. for the readout circles).

The surrounding circles were spotted with 0.2 μL of green, light green, blue, light blue, orange, red, brown and pink dyes that represent the reagents of potential bioassays: Yellow dye (2.0 μL) was placed in the centre circle to simulate the sample volume, and the resulting colour change in each read-out zone, represents successful test readouts. Thus, the central circle receives the sample fluid, which flows to the surrounding eight test circles to produce eight different color changes. This demonstrates the utility of the present fabrication technique to create compact and microscale features in paper (i.e. a microfluidic device) for use with micro-samples.

Glucose Test with Small Volume of Urine Sample—

To demonstrate the efficacy of the fabrication technique with a bioassay, a glucose oxidase (GOx) assay was conducted using only 2 μL of artificial urine sample was performed. The same layout as described for the dye test was used. A well-established colorimetric detection technique was used as described above. The reagents were initially colourless and after the urine sample is placed in the sample circle the test readout zones change to a dark brown colour within 5 minutes of sample placement indicating the presence of glucose. The intensity of the brown colour depends on the concentration of the glucose in the urine sample. This demonstrates the successful use of the present microfluidic device in a bioassay using a micro-sample (i.e. 2 μL of sample). In practice, the eight readout zones could contain different reagents for a variety of tests.

### Conclusions

Thus, a simple fabrication technique has been developed that enables patterning of compact and microscale features on paper-based microfluidic devices with the use of a laser cutting machine. The materials required for the fabrication are inexpensive, readily available and easy to use in the fabrication process. In addition, this technique can be incorporated in the mass production of μPADs. This technique enables miniaturizing of μPADs so that small sample volumes can be used and thus reduces the amount of materials used in the device, reduces the chemical reagent volumes required for bioassays, reduces the packaging cost, and results in inexpensive μPADs for global diagnostic and environmental testing applications.

Devices with channel barriers of width of 39±15 μm were prepared that were capable of restricting fluid flow across the barrier. Channels with a width of about 100 μm were found to permit fluid flow in the chromatography paper used. A successful dye test and glucose test were conducted with a device with eight readout zones using only 2 μL of sample fluid volume to demonstrate that the present technique may be used to create a device capable of creating compact and microscale bioassays.

### Example 2

Microfluidic devices comprising various geometries of hydrophilic regions were made as described below.

A two-way μPAD architecture flowing in three dimensions (3D), made in aluminum foil-backed Whatman 1 chromatography paper via subtractive patterning using a laser, was prepared. The μPAD comprised a first subtractive pattern (to yield a first fluid flow region), and a second subtractive pattern (to yield a second fluid flow region) perpendicular to the first on either side of the first subtractive pattern. The fluid flow portions of the second subtractive patterns were connected underneath the first fluid flow region via an absorbent substrate channel comprising cellulose paste. Two different colored dye samples were applied to each of the first and second fluid flow regions. A red sample applied to one side of the second fluid flow region passed underneath a blue sample applied to the first fluid flow region and was observed on the other side of the second fluid flow region without mixing with the blue sample in the first fluid flow region.

A four-way μPAD architecture, flowing in three dimensions (3D), was prepared using aluminum foil backed Whatman 1 chromatography paper via subtractive patterning using a laser. The subtractive patterning produced four fluid flow regions, each comprising cellulose paste bridges passing above or underneath the other fluid flow regions. To each fluid flow region, a different colored dye sample was applied. Fluid flow was observed to be maintained within each fluid flow region without mixing of colored dyes. This example illustrates the complexity of PAD architecture that is possible with the present device.

Another two-way μPAD architecture made in aluminum foil and polyester-backed nitrocellulose was prepared via subtractive patterning using a laser to flow two samples along separate fluid flow path lengths, one of which was a straight path, and the other of which was a serpentine path. The polyester backing to nitrocellulose is not impermeable to the laser and is damaged by the laser, but the architecture remains in place via the adhesive holding the materials to the impermeable aluminum foil, maintaining the etched boundaries and preventing leakage. Dyed samples applied to each path length were shown to flow along the path, including flow along the serpentine path length.

A three-way multiplex μPAD architecture made in aluminum foil and polyester backed nitrocellulose paper was prepared via subtractive patterning using a laser to make a multiplex color assay. The subtractive patterning provided a sample circle fluidly connected via 3 arms to 3 distinct test circles comprising bromophenol blue, glucose oxidase, and potassium iodide, respectively, for colorimetric detection of sample. A synthetic serum sample added to the sample circle of the μPAD flowed to the test circles, changing the colors of the three test sites.

The foregoing illustrates that utility of the present μPAD having different geometries and 2 or more dimensions.

In the example embodiments, channel barriers were created with widths of 36±13 m and 39±15 μm that were capable of restricting fluid flow across the barrier. As well, channels with a width of 128±30 μm were generated. A successful dye test and glucose test were performed with eight readout zones using only 2 μL of sample fluid volume to demonstrate that the assembly and fabrication method of the present invention is capable of creating compact and microscale bioassays.

### Example 3

In this experimental study, the smallest possible feature sizes that will enable fluid flow were studied in five different types of paper: (i) Whatman 1 Chr chromatography paper (1 Chr), (ii) Whatman 3 MM Chr chromatography paper (3 MM Chr), (iii) Whatman regenerated cellulose membrane 55 (RC-55), (iv) Whatman filter paper grade 50 (FP-50), and (v) Amershan Protran 0.45 nitrocellulose membrane (NC).

Materials—

Whatman 1 Chr chromatography paper (1 Chr), Whatman 3 MM Chr chromatography paper (3 MM Chr), Whatman regenerated cellulose membrane 55 (RC-55), Whatman filter paper grade 50 (FP-50), and Amershan Protran 0.45 nitrocellulose membrane (NC). All paper types are manufactured by GE healthcare. Allura Red AC of dye content 80% was purchased from Sigma-Aldrich (Oakville, Ontario, Canada) and aluminum foil (as above) was purchased from UOIT central stores, Oshawa, Ontario. A roll of positionable mounting adhesive film 568 by 3M™ was purchased from Amazon.ca.

Fabrication of Small-Scale Features—

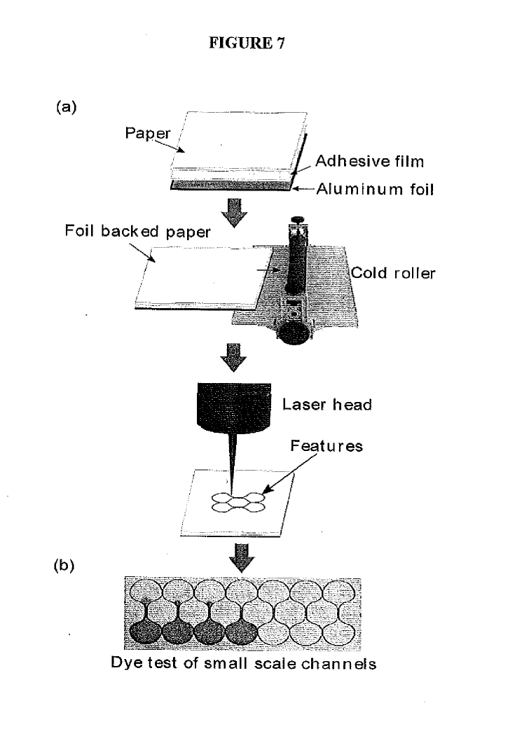

Micro-scale features were fabricated in the five different paper materials using the method as described in Example 1. Modifications to the previous method include use of a positionable mounting adhesive film (3M™) in place of the double sided tape and use of a manual cold laminator (manual vinyl film mounting Cold Laminator, sold by ASC365 International Ltd., Amazon.ca) to bond the layers, as shown in FIG. 7. A 30 W CO₂ laser (Speedy 100, Trotec) was used to create the barriers around the features through removal of the hydrophilic paper material. Channels of different widths were fabricated, from 240 μm down to 140 μm line-to-line design width, which is the distance between the lines that are drawn in Inkscape and input into the laser to determine the path followed by the laser beam, with an interval of 20 μm. The actual widths of the channels that result on the paper material after cutting by the laser are smaller than the design width, and actual resulting widths are reported herein.

To establish independence between the fabrication method and the smallest possible feature sizes in commercially available paper types, experiments were conducted using Chr 1 paper without a foil backing in which channels were fabricated by laser cutting. For this fabrication, the paper was cut without any adhesive or foil backing and the channel was held in place by leaving a connection to the main paper sheet, thus not fluidically isolating the channel from the rest of the paper sheet with the hydrophobic barrier, following a method similar to that described by Zie et al. The Analyst 2012, 138 (2), 671-676.

Determination of Smallest Possible Features that Enable Fluid Flow—

Channels of different widths (ranging from 240 μm to 140 μm of line-to-line design widths with an interval of 20 μm) were fabricated from each material in such a way that each channel connects two reservoir circles with a final shape that resembles a dumbbell, as shown in FIG. 7. A pipette was used to place 2 μL of red dye (0.5 g/L—Allura Red) on one of the circles. A successful channel was deemed to be one where the dye wicks through to the opposite circle based on observation. The actual widths of the channel were measured with a microscope (OMAX 40×-1600× professional EPI-fluorescence trinocular biological microscope with IOMP USB digital Camera, sold by MicroscopeNet Canada, Amazon.ca).

Determination of Dye Flow Speed Through the Small-Scale Features—

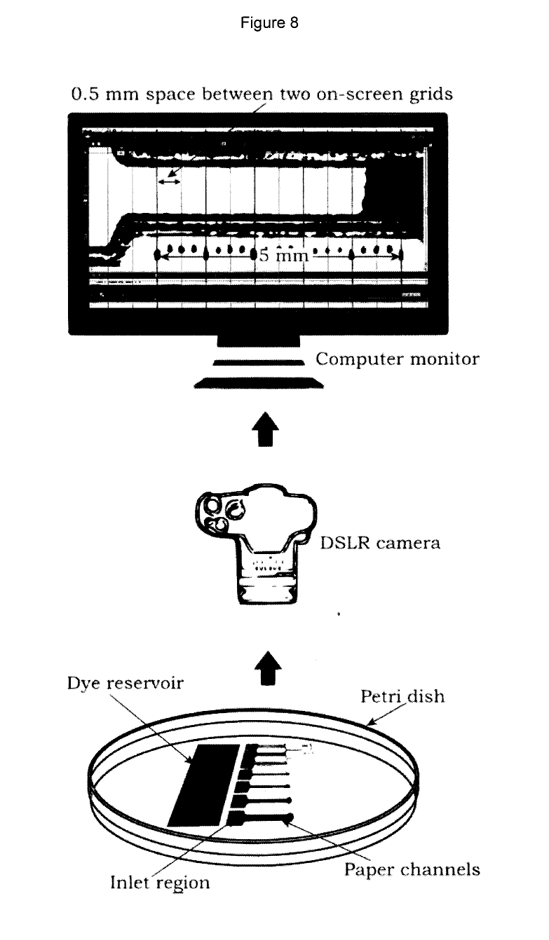

Dye flow speed was measured through small-scale channels of different widths fabricated from three of the different paper types: Chr-1, 3 MM Chr and RC-55. A schematic of the experimental procedure is shown in FIG. 8. Each paper channel was located on a petri dish and fed from a triangular paper reservoir that contained an excess volume of dye (0.5 g/L, Allura Red) on it. The surface of the petri dish is hydrophobic which causes the dye to move directly to the channel without spreading along the petri dish. The tip of the reservoir connects with an inlet region of each channel and provided an unlimited fluid supply for continuous flow through each channel. The petri dish was covered with its lid at the moment when the tip of the reservoir was brought in contact with the inlet region of the channel to reduce the effect of the evaporation loss on the system. The flow was recorded with a DSLR camera (Nikon D5200 with Nikon Af-s Dx Micro 40 mm F2.8G lens) which was connected with a PC to observe the flow on the monitor. A 5 mm scale with 250 μm tick marks was cut with the laser along each channel to measure the time required by the liquid front to travel a specific distance. To align the liquid front with the printed tick marks an on-screen, a grid software (MB-Ruler) was used that generates grids with a precise tick mark spacing. VSDC video editing software was used to measure the time required by the liquid front to travel between tick marks with millisecond timing.

### Results

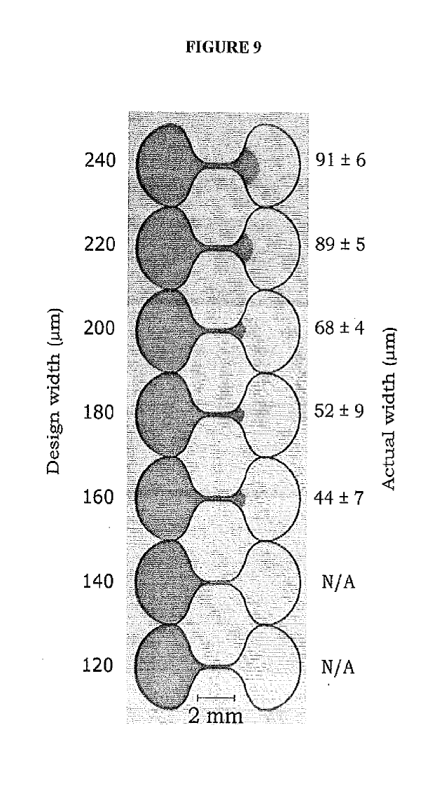

To determine the smallest feature size in certain paper-based devices that enables fluid flow, fluid flow along a 1 mm long channels which connect two reservoirs and which vary in width was measured in different paper types as shown in FIG. 9. The tests were repeated in triplicate to establish repeatability and the results are summarized in Table 1.

TABLE 1

| | Paper | Smallest |
|---|---|---|
| Type | thickness | Channel width |
| FP50 | 115 | 139 ± 8 |
| 3 MM | 340 | 130 ± 11 |
| Chr 1 | 180 | 106 ± 11 |
| (w/o foil) | | |
| Chr 1 | 180 | 103 ± 12 |
| RC55 | 75 | 45 ± 6 |
| NC | 135 | 24 ± 3 |

As shown in Table 1, the narrowest channel width to permit fluid flow was formed in nitrocellulose membrane (NC). Comparison of the two fabrication techniques (with and without foil backing) show that the smallest width for successful fluid flow in each device is similar, thus demonstrating independence from the specific fabrication method.

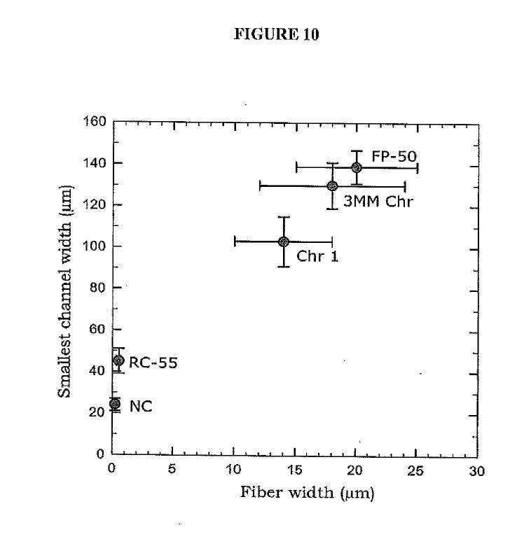

To understand which parameters influence the smallest features that enable flow in paper-based devices, the present data was correlated with some of the physical properties of the paper types. A correlation between fiber width and the narrowest possible channel width for successful fluid flow was observed. The average fiber width was determined from the diameter of the fiber observed in SEM images. The fiber width of the five different paper types was plotted against the smallest possible channel width as shown in FIG. 10. The plot shows that, generally, the smaller the fiber width, the smaller the channel width that will permit fluid flow. Thus, in some cases, channel widths of less than 100 μm are possible in paper substrates with average fiber widths of less than 5 μm, less than 2 μm, or less than 1 μm, e.g. 0.1-0.5 μm. Paper substrates comprising average fiber widths greater than 5 μm, such as 10-20 μm, may yield channels of greater than 100 μm, e.g. 110 μm, 120 μm, 130 μm, 140 μm, 150 μm and greater.

It was also determined that for successful fluid flow through a paper channel, the fiber structure should be continuously linked along the channel pathway to ensure that the fluid is wicked along by capillary forces. A channel fails to carry liquid when the fiber network along the channel becomes disconnected, e.g. by fibers which are loose or destroyed. SEM images confirm that unsuccessful channels comprise a fiber network that is discontinuous as the channel widths are made too small. Therefore, the paper types with smaller fiber widths are capable of having continuous fiber networks along smaller channels (e.g. <100 μm), while paper with larger fiber widths maintain continuous fiber networks in channels which are larger (e.g. >100 μm).

To examine the flow behavior through micro-scale features in paper-based devices, experiments using Chr 1, 3 MM Chr, and RC-55 were conducted. The time required for fluid to travel 5 mm with intervals of 0.5 mm was determined. The line-to-line design widths and corresponding actual widths for the three paper types used in these experiments are summarized in Table 2.

TABLE 2

| Chr-1 | | 3 MM Chr | | RC-55 | |
|---|---|---|---|---|---|
| Design | Actual | Design | Actual | Design | Actual |
| width | width | width | width | width | width |
| (μm) | (μm) | (μm) | (μm) | (μm) | (μm) |
| — | — | — | — | 200 | 58 ± 7 |
| 300 | 193 ± 16 | 300 | 220 ± 13 | 300 | 165 ± 7 |
| 400 | 285 ± 16 | 400 | 317 ± 13 | 400 | 255 ± 7 |
| 500 | 390 ± 16 | 500 | 410 ± 13 | 500 | 364 ± 7 |
| 600 | 482 ± 16 | 600 | 514 ± 13 | 600 | 451 ± 7 |
| 1100 | 971 ± 16 | 1100 | 982 ± 13 | 1100 | 930 ± 7 |

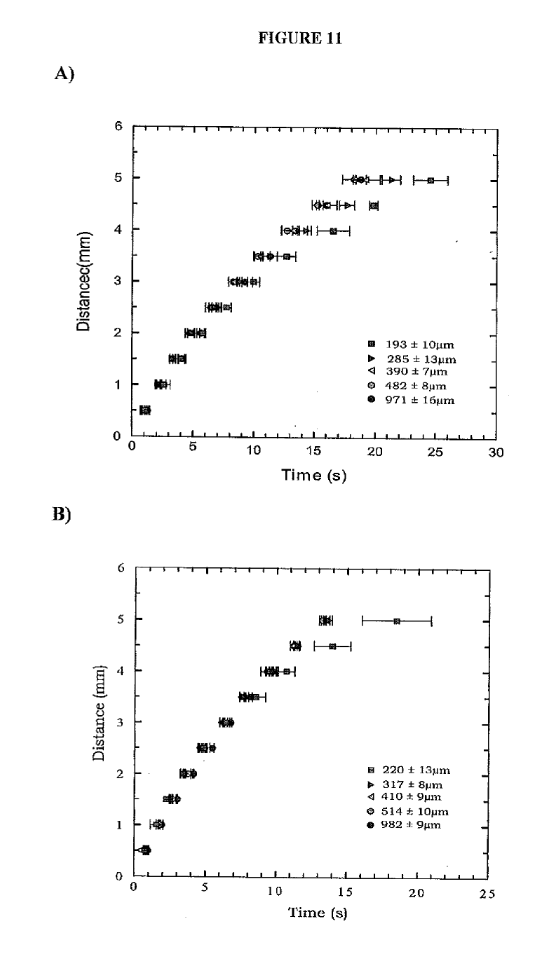

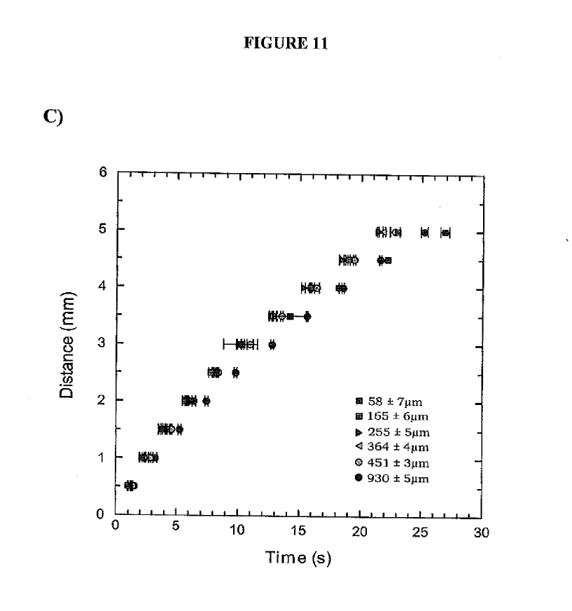

FIG. 11 (A-C) show the travel time of the liquid front through various channel widths for Chr-1, 3 mm Chr and RC-55, respectively. FIG. 11A shows that there is little observable change in flow speed for the varying widths in Chr-1, except at the two smallest widths where the flow was observed to slow down. FIG. 11B shows the same trend for 3 MM Chr, except the only observable variation is for the smallest channel width, and relative to the Chr 1 experiments, the flow is faster through the 3 MM Chr paper. FIG. 11C shows that RC-55 also follows the same trend as 3 MM Chr, with flow speeds that are closer to Chr 1. Thus, generally, flow speed increases with channel width.

The above-described embodiments of the invention are intended to be examples of the present invention and alterations and modifications may be effected thereto, by those of skill in the art, without departing from the scope of the invention, which is defined by the claims appended hereto.

References referred to herein are incorporated by reference.

What is claimed is:

1. A microfluidic device comprising an absorbent substrate layer affixed to a layer impermeable to an etching tool, wherein portions of the substrate layer are removed to form at least one subtractive pattern which directs fluid flow within the device.

2. The microfluidic device of claim 1, wherein the substrate layer comprises paper, glass fiber, nitrocellulose, polymers, or plastics.

3. The microfluidic device of claim 1, wherein the substrate layer is hydrophilic.

4. The microfluidic device of claim 1, wherein the substrate layer Is paper.

5. The microfluidic device of claim 1, wherein the impermeable layer is a metallic foil, plastic or a polymer.

6. The microfluidic device of claim 1, wherein the Impermeable layer is aluminum foil.

7. The microfluidic device of claim 1, wherein the impermeable layer is impermeable to a laser.

8. The microfluidic device of claim 1, wherein the substrate layer is affixed to the impermeable layer by an adhesive layer.

9. The microfluidic device of claim 8, wherein the adhesive layer is a tape, glue or wax.

10. The microfluidic device of claim 1, wherein the subtractive pattern is a hydrophobic barrier to fluid flow which encompasses a hydrophilic fluid flow region.

11. The microfluidic device of claim 10, wherein the hydrophobic barrier comprises a width of between 25-80 μm.

12. The microfluidic device of claim 10, wherein the hydrophilic fluid flow region comprises a sample zone and at least one detection zone connected via a channel.

13. A method of manufacturing a microfluidic device, the method comprising:
forming a substrate assembly, the substrate assembly comprising a substrate layer affixed to an impermeable layer; and cutting away portions of the substrate layer using an etching device to form one or more subtractive patterns on the substrate assembly.

14. The method of claim 13, wherein the etching device is a laser.

15. The method of claim 13, wherein the substrate layer comprises paper, glass fiber, nitrocellulose, polymers, or plastics.

16. The method of claim 13, wherein the impermeable layer Is a metallic foil, plastic or a polymer.

17. The method of claim 13, wherein the substrate layer is affixed to the impermeable layer with an adhesive layer which is a tape, glue or wax.

18. The method of claim 13, wherein the one or more subtractive patterns form one, two or three dimensional flow systems.

19. The method of claim 13, wherein the subtractive pattern is a hydrophobic barrier to fluid flow that encompasses a hydrophilic fluid flow region.

20. A system for manufacturing microfluidic devices, the system comprising:
a feed assembly, for directing a layer of substrate and an impermeable layer towards a combining assembly for affixing the substrate to the impermeable layer using an adhesive to form an assembled substrate;
an etching device for cutting away portions of the substrate layer to form one or more subtractive patterns on the assembled substrate; and
a cutting device for cutting the assembled substrate into one or more microfluidic devices.

21. A multi-dimensional microfluidic device comprising 2 or more devices as defined in claim 1 connected via channels which permit fluid flow from one device to another.
